(19) United States
(12) Patent Application Publication
(10) Pub. No.: US 2018/0192987 A1
(43) Pub. Date: Jul. 12, 2018

(54) ULTRASOUND SYSTEMS AND METHODS FOR AUTOMATIC DETERMINATION OF HEART CHAMBER CHARACTERISTICS

(71) Applicant: KONINKLIJKE PHILIPS N.V., EINDHOVEN (NL)

(72) Inventors: IVAN SALGO, EINDHOVEN (NL); RAJESH PANDA, EINDHOVEN (NL); ASHOK TEWARI, EINDHOVEN (NL)

(21) Appl. No.: 15/741,513

(22) PCT Filed: Jul. 7, 2015
(86) PCT No.: PCT/IB2016/054000

§ 371 Date: Jan. 3, 2018

Related U.S. Application Data
(60) Provisional application No. 62/189,444, filed on Jul. 7, 2015.

(51) Int. Cl. A61B 8/04; A61B 8/08; A61B 8/00

(52) U.S. Cl. CPC A61B 8/04; A61B 8/0883; A61B 8/5223; A61B 8/5207; A61B 8/463

(57) ABSTRACT

Ultrasound systems and methods for automatically determining heart chamber characteristics are provided. The systems and methods of the present invention, for example, can automatically determine chamber pressures and volumes of a patient's heart using a transthoracic or transesophageal ultrasound probe imaging the heart in 2D and/or 3D. Chamber pressures determined include the static pressure of the left ventricular end diastolic pressure, which can be used to diagnose whether a patient is suffering from congestive heart failure.

### BACKGROUND

Current approaches for diagnosing congestive heart failure are limited. Typically, patients suffering from congestive heart failure visit a hospital complaining of difficult breathing along with other symptoms associated with fluid in the their lungs. Such symptoms are associated with many different types of conditions, such as chronic obstructive pulmonary disorder (COPD), pneumonia, or congestive heart failure (CHF), and it can be difficult to diagnose the actual condition of the patient. Physicians can obtain X-rays, for example, to identify whether fluid is present in the lungs, and further monitor weight loss due to loss of fluids after administering diuretics to the patient. Cocktails of antibiotics and steroids can also be administered. If a patient is seen as losing a sufficient amount of water weight after taking the diuretics, physicians will typically release them assuming that the issue has been remedied and patient will continue to improve from further loss of fluid due to the diuretics. Unfortunately, this approach is not effective for congestive heart failure, and such patients have to return to the hospital within the coming weeks because the underlying condition was not remedied, only the indirect symptoms. While it would be ideal to limit the number of returning patients suffering from CHF, existing procedures for accurate diagnosis involve invasive interventional procedures that involve using a catheter to determine pressures in the patient's heart. Of course, invasive catheter-based procedures are expensive and can be potentially dangerous with an increased chance of infection for the patient.

Accordingly, there is a need for better methods and systems to non-invasively diagnose congestive heart failure. The present invention addresses this need and more.

### SUMMARY

In some embodiments, the present invention provides ultrasound systems for automatically determining a static pressure in at least one heart chamber of a patient. The ultrasound systems can include an ultrasound probe adapted to scan the at least one heart chamber, an image processor coupled to the ultrasound probe and adapted to produce an ultrasound image of the at least one heart chamber, a segmentation processor configured to identify a boundary of the at least one heart chamber in the ultrasound image and determine a spatial characteristic of the at least one heart chamber, a waveform processor configured to generate a waveform representing changes of the spatial characteristic over a time period, and a display configured to display a representation of the static pressure in the at least one heart chamber of the patient based at least in-part on the waveform.

### BRIEF DESCRIPTION OF THE DRAWINGS

FIG. 1 is a schematic view of an embodiment of an ultrasound imaging system according to an example embodiment of the invention.

FIG. 2 is a method for automatically calculating a static pressure of a heart chamber, according to an example embodiment of the invention.

FIG. 3 is a display of an ultrasound system, according to an example embodiment of the invention.

FIG. 4 shows a display of an ultrasound system, according to an example embodiment of the invention.

FIG. 5 illustrates a display of an ultrasound system, according to an example embodiment of the invention.

FIG. 6 illustrates a display of an ultrasound system, according to an example embodiment of the invention.

### DETAILED DESCRIPTION

In the following detailed description, for purposes of explanation and not limitation, illustrative embodiments disclosing specific details are set forth in order to provide a thorough understanding of an embodiment according to the present teachings. However, it will be apparent to one having ordinary skill in the art having had the benefit of the present disclosure that other embodiments according to the present teachings that depart from the specific details disclosed herein remain within the scope of the appended claims. Moreover, descriptions of well-known apparatus and methods may be omitted so as to not obscure the description of the illustrative embodiments. Such methods and apparatus are within the scope of the present teachings.

The following detailed description is therefore not to be taken in a limiting sense, and the scope of the present system is defined only by the appended claims. The leading digit(s) of the reference numbers in the figures herein typically correspond to the figure number, with the exception that identical components which appear in multiple figures are identified by the same reference numbers. Moreover, for the purpose of clarity, detailed descriptions of certain features will not be discussed when they would be apparent to those with skill in the art so as not to obscure the description of the present system.

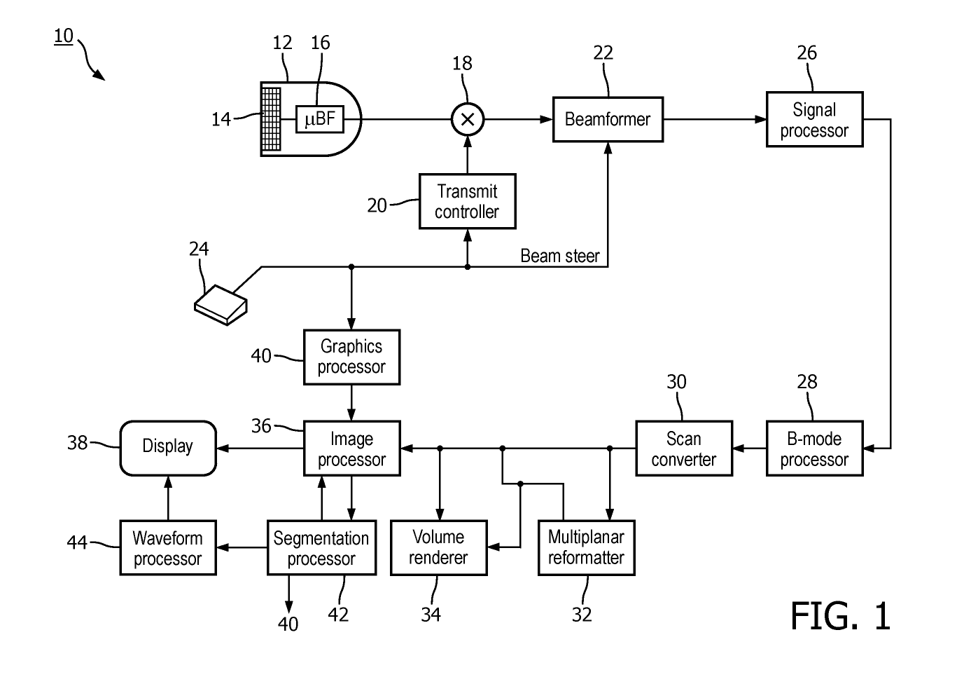

Referring to FIG. 1, an ultrasound imaging system 10 constructed in accordance with the principles of the present invention is shown in block diagram form. The block diagram form shows a representation of hardware structures in conventional ultrasound systems that can be configured via hardware and/or software to operate according to the methods and algorithms described further herein. In the ultrasonic diagnostic imaging system of FIG. 1, an ultrasound probe 12 includes a transducer array 14 for transmitting ultrasonic waves and receiving echo information. A variety of transducer arrays are well known in the art, e.g., linear arrays, convex arrays or phased arrays. The array can be fabricated as a one dimensional (1D) or a two dimensional (2D) array of transducer elements. Either type of array can scan a 2D plane and the two dimensional array can be used to scan a volumetric region in front of the array. The transducer array 14, for example, can include a two dimensional array (as shown) of transducer elements capable of scanning in both elevation and azimuth dimensions for 2D and/or 3D imaging. The transducer array 14 is coupled to a microbeamformer 16 in the probe 12 which controls transmission and reception of signals by the transducer elements in the array. In this example, the microbeamformer is coupled by the probe cable to a transmit/receive (T/R) switch 18, which switches between transmission and reception and protects the main beamformer 22 from high energy transmit signals. Microbeamformers are capable of at least partial beamforming of the signals received by groups or “patches” of transducer elements as described in U.S. Pat. No. 5,997,479 (Savord et al.), U.S. Pat. No. 6,013,032 (Savord), and U.S. Pat. No. 6,623,432 (Powers et al.), each of which is incorporated by reference herein. In some embodiments, the T/R switch 18 and other elements in the system can be included in the transducer probe rather than in a separate ultrasound system base. The transmission of ultrasonic beams from the transducer array 14 under control of the microbeamformer 16 is directed by the transmit controller 20 coupled to the T/R switch 18 and the beamformer 22, which receives input from the user's operation of the user interface or control panel 24. One of the functions controlled by the transmit controller 20 is the direction in which beams are steered. Beams may be steered straight ahead from (orthogonal to) the transducer array, or at different angles for a wider field of view. The partially beamformed signals produced by the microbeamformer 16 are coupled to a main beamformer 22 where partially beamformed signals from individual patches of transducer elements are combined into a fully beamformed signal.

The beamformed signals are coupled to a signal processor 26. The signal processor 26 can process the received echo signals in various ways, such as bandpass filtering, decimation, I and Q component separation, and harmonic signal separation. The signal processor 26 may also perform additional signal enhancement such as speckle reduction, signal compounding, and noise elimination. The processed signals are coupled to a B mode processor 28, which can employ amplitude detection for the imaging of structures in the body. The signals produced by the B mode processor are coupled to a scan converter 30 and a multiplanar reformatter 32. The scan converter 30 arranges the echo signals in the spatial relationship from which they were received in a desired image format. For instance, the scan converter 30 may arrange the echo signal into a two dimensional (2D) sector-shaped format, or a pyramidal three dimensional (3D) image. The multiplanar reformatter 32 can convert echoes which are received from points in a common plane in a volumetric region of the body into an ultrasonic image of that plane, as described in U.S. Pat. No. 6,443,896 (Detmer). A volume renderer 34 converts the echo signals of a 3D data set into a projected 3D image as viewed from a given reference point, e.g., as described in U.S. Pat. No. 6,530,885 (Entrekin et al.) The 2D or 3D images are coupled from the scan converter 30, multiplanar reformatter 32, and volume renderer 34 to an image processor 36 for further enhancement, buffering and temporary storage for display on an image display 38. The graphics processor 40 can generate graphic overlays for display with the ultrasound images. These graphic overlays can contain, e.g., standard identifying information such as patient name, date and time of the image, imaging parameters, and the like. For these purposes the graphics processor receives input from the user interface 24, such as a typed patient name. The user interface can also be coupled to the multiplanar reformatter 32 for selection and control of a display of multiple multiplanar reformatted (MPR) images.

As provided herein, the image processor 36 produces scanline data of an image which can be stored in image data memory, e.g., in random access memory of the ultrasound system. The segmentation processor 42 can receive the image data from the image processor and then apply segmentation algorithms to segment features (e.g., a heart chamber) in the images. For example, a first, starting point image of a sequence of heart images can be analyzed by border detection of a heart chamber by the segmentation processor as described more fully below. When the border is defined in this first image its location is tracked through subsequent images. For display, the initially defined border and the border in subsequent images are drawn by the graphics processor 40. The ultrasound images of the sequence can be further converted to the desired display format (e.g., sector, linear, 3D, etc.) and displayed with graphically produced borders over the defined border locations in the ultrasound images. The image with its graphic border overlay can be stored in a Cineloop memory 460, and the images can then be displayed on the display 38.

In some embodiments, specific points on the identified borders of the successive images can be tracked from the starting anatomical positions of the points by a speckle pattern produced by the local tissue at the image locations of the points. The segmentation processor 42 can, for example, identify regions of pixels around the reference points in the adjacent myocardium. The speckle patterns of these pixels are saved and compared with speckle patterns in the same regions of the successive images and the speckle patterns matched by block matching, as described in U.S. Pat. No. 6,442,289 (Olsson et al.), which is incorporated by reference herein. The difficulty and precision of the matching is determined by establishing a maximum correlation for the matching. The reference point locations in the images are thus tracked from image to image by following the speckle patterns around the points. When the segmentation processor 42 locates the reference points in a new image the reference point locations are coupled to the graphics processor 40, the border can be redrawn using the newly identified point locations, and a graphic overlay can be produced for the new image. The new image and its graphic overlay are scan converted and displayed on the display 38. The segmentation processor 42 can also be programmed to perform a variety of the other segmentation algorithms and to track other image characteristics. For instance, the movement of specific anatomical features may be tracked. As another example, tissue texture may be tracked. It will also be appreciated that the targeted characteristics may be tracked in either pre-scan converted or post-scan converted image data. Another example of border tracing with the segmentation processor can be applied as described, e.g., in U.S. Pat. No. 7,794,398 (Salgo), which is incorporated by reference herein. A wide variety of automatic or semi-automatic border detection processes may also be used, including those described in U.S. Pat. No. 6,491,636 (Chenal et al.); U.S. Pat. No. 6,346,124 (Geiser et al.); and U.S. Pat. No. 6,106,465 (Napolitano et al.), which are incorporated by reference herein.

As further shown in FIG. 1, the segmentation processor 42 can be coupled to a waveform processor 44. The waveform processor 44 includes a microprocessor, or integrated circuit or other hardware chip-based device that can be programmed with software to receive data, operate on such data, and output data. For example, the waveform processor 44 receives data from the segmentation processor 42 to generate data that can be used to plot changes in the spatial characteristic of the heart chamber overtime. For example, the segmentation processor 42 can identify a boundary around the heart chamber (e.g., a left ventricle) and calculate a volume of the chamber. Images collected in a Cineloop for example over a period of time can be processed in sequence to determine the volume of the heart chamber in each of the images, and then tracked over time with the waveform processor 44 which can generate a plot of the volume as a function of time. This data of the volume as function of time can be used by the software embedded on the ultrasound system to calculate a variety of information about the heart and/or heart chamber.

Residing in the software of the ultrasound system is also a variety of algorithms for calculating information about the patient's heart. In some embodiments, the algorithms for calculating chamber pressures can rely on statistical data that is stored on the ultrasound system and compared to real data produced during a scanning procedure. Statistical catheter-based studies performed on a set of patients provide values of static pressure values that can be used to diagnose whether a patient is suffering from congestive heart failure. If, for example, the static pressure identified using the methods and systems herein is above 10 or 15 mm Hg, then a user reading such a value can compare to the statistical data to identify whether congestive heart failure is likely.

Other algorithms can also be used. For example, several image views of the heart and/heart chambers can be acquired and stored on the ultrasound system. In addition to the views, 2D and/or 3D images can be stored as a series of images and analyzed over a period of time to track motion in the heart and/or heart chamber. The motion tracking can be used to produce quantified values of cardiac performance such as ejection fraction and cardiac output. A combination of the views and quantified values can be used to further calculate the static pressure of a heart chamber, such as the left ventricular end diastolic pressure. Other formulas for determining static pressure values can include plotting a left atrial volume curve over time to identify various points in the curve to be used for calculating static pressures. An example algorithm for this approach uses the kinetics-tracking (KT) index, which is described in Kawasaki et al., Noninvasive estimation of pulmonary capillary wedge pressure using speckle tracking echocardiography in patients with preserved or reduced ejection fraction, European Heart Journal, Aug. 1, 2013 and Kawasaki et al., A novel ultrasound predictor of pulmonary capillary wedge pressure assessed by the combination of left atrial volume and function: A speckle tracking echocardiography study, Journal of Cardiology, Dec. 26, 2014, both of which are incorporated by reference herein. The KT index uses left atrial (LA) emptying function (EF) and volume (LAV) assessed by speckle tracking echocardiography and is represented as follows: log₁₀ (active LAEF/minimum LAV index). Along the left atrial volume curve, there is a maximum volume, a pre-atrial contraction volume, and a minimum volume. It is envisioned herein that the ultrasound systems and methods of the present invention can apply these techniques in determining heart chamber pressures, such as left ventricular end diastolic pressure.

It is noted that the robustness of the pressure measurements can rely heavily on the underlying volume measurements of the heart chambers. Therefore, approaches for calculating an accurate volume of heart chambers, e.g., an LA or LV or both is provided herein. In some embodiments, the present invention provides systems and methods for making more accurate LA and/or LV measurements for, e.g., calculating pressures. For example, the present invention includes using template matching (e.g., pattern matching) to generate seed points for generating automatic regions of interest (ROIs) in ultrasound images of a patient's heart. In some aspects, template matching can be used to identify two basal points and one apical point in the heart shown in an ultrasound image and then use those identifications to automatically generate an ROI. Templates used for matching patterns in the images can be stored on the system in memory and searched to identify templates that include two basal points and one apical point. Once the template is identified, the system recognizes the view of the image that has been generated, e.g. an apical view. Because the system knows the view, it can further select a particular template that corresponds to that view and use a view-specific model to improve boundary detection of the heart chamber. The template matching approach is particularly useful for adapting to different shapes of the heart chambers for different patients. For example, one patient's LA may be longer and thinner than another patient and/or have additional bulges or ridges in the chamber that need to be accurately accounted for in the volume calculation for the chamber.

After template matching is performed, the present invention can further include improved edge detection for the tissue boundary in the heart chamber being measured, e.g., the LA or the LV. Edge detection can be carried out using techniques known in the art such as the technique disclosed in WO2004092766, which is incorporated herein by reference in its entirety. Once the boundary edge of the tissue is accurately determined with the combination of the template matching and edge detection, an optical flow methodology can be used to track the boundary through the heart cycle, thereby calculating the waveform for use to determine pressure, such as the static pressure. Optical flow methods of the present invention are specifically formulated for tracking each heart chamber. In one embodiment, the optical flow methodology uses the expected position of the LA in a particular view, e.g., the apical view that is acquired by a user. Based on the position in the image and the tissue characteristics of the LA, a level of regularization that includes constraints for movement in the algorithm are optimized. In contrast, the LV has more muscle and moves differently than the LA, and therefore different regularization and constraints are used in the optical flow methodology for tracking the LV vs. the LA.

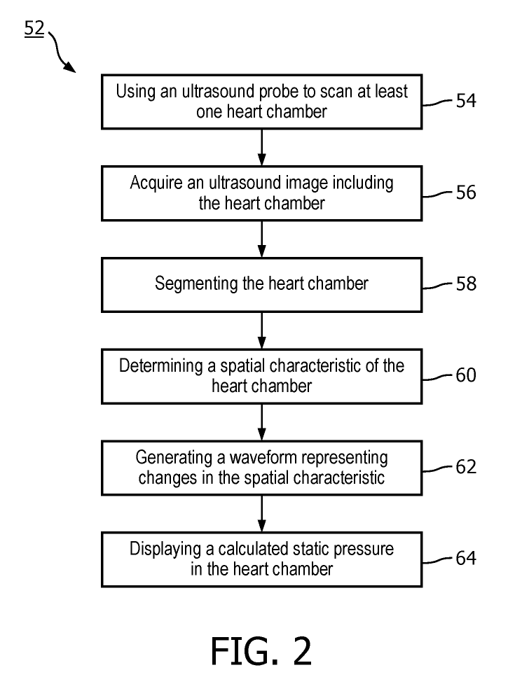

FIG. 2 shows an example method 52 of the present invention. The methods can include using an ultrasound probe described herein to scan at least one chamber of a heart (Step 54). For example, the ultrasound probes can be positioned on a patient to scan in 2D or 3D the left ventricle of the patient's heart. The method further includes acquiring an ultrasound image including the heart chamber (Step 56). A Cineloop of 2D images can be acquired as well. In some instances, a four-chamber view, five-chamber view, three-vessel view, a tracheal view, or a combination thereof can be acquired. Based on the methods described herein, the acquired images can be used to segment the heart chamber using the ultrasound systems of the present invention (Step 58). With the segmentation data, a boundary around the heart chamber can be determined and used to calculate dimensions in 2D or 3D depending on the image data being segmented. Accordingly, the method includes determining a spatial characteristic of the heart chamber (Step 60). Spatial characteristics that can be determined based on the segmentation, include but are not limited to volume, perimeter, area, curved length, and cavity length. In some instances, strain information of the heart tissue can also be acquired and tracked. The next step of the method includes generating a waveform representing changes in the spatial characteristic (Step 62). For example, the volume of the heart chamber can be tracked overtime and plotted as a waveform to be displayed (e.g., as a graph or chart) for viewing on the display of the ultrasound system (Step 64). Based on the image data and/or waveform generated by the ultrasound system, a static pressure (e.g., of the left ventricle) can be calculated using the method described herein. Thus, the methods can include displaying a calculated static pressure in the heart chamber for consideration. In some instances, the static pressure will be used to diagnose or indicate a possibility of congestive heart failure.

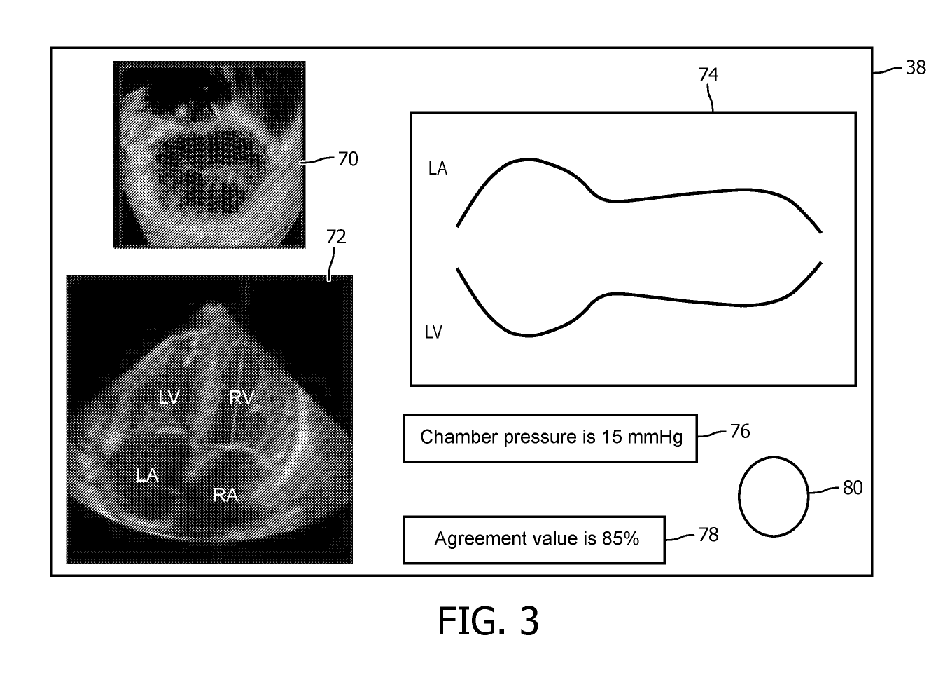

In some aspects of the present invention, ultrasound systems and methods can automatically determine heart chamber pressures and provide a variety of displays to a user to confirm accuracy of the images used for the analysis as well as the value calculated for the heart chamber pressures. FIG. 3 shows an example display configured to display images and waveforms generated by the waveform processor. The display 38 can show, for example, an image of top down view of a right ventricle 70 in combination with a side view of the right ventricle 72. As shown the segmentation processor can be used to identify tissue boundaries within the chamber and provide, e.g., perimeter and/or volume information about the selected heart chamber. In some embodiments, LA/LV or RA/RV border detection of two chambers can be used to compute filling pressure in the heart. In addition to the images, the display is further configured to show the waveforms generated by the waveform processor. As shown in FIG. 3, a spatial characteristic (e.g., volume) of a left atrium can be plotted in combination with a spatial characteristic (e.g., volume) of a left ventricle over time 74. In some embodiments, the display can be further configured to display the calculated static pressure 76, as well as an indicator 80 providing a user a quick way to determine whether the calculated value is acceptable or whether a new scan or calculation should be performed. The indicator 80 can include, e.g., a color code (such as green, red or yellow). An agreement value 78 can also be generated with the ultrasound system of the present invention. The agreement value shows a relative comparison between the two waveforms of connected heart chambers. For example, the filling and ejection profiles of the left atrium and the left ventricle can be compared by the ultrasound system using a variety of approaches, such as convolution and correlation, which are both well known to one of ordinary skill in the art.

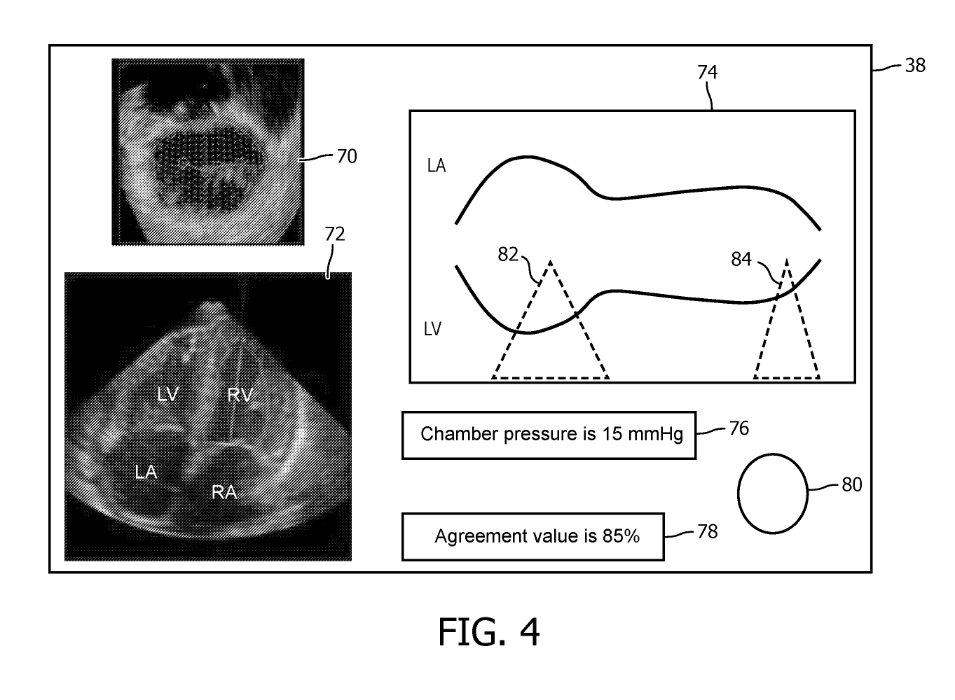

In addition to the images and waveforms displayed for a user to check and confirm accuracy of a heart chamber calculation, such as calculation of the left ventricle end diastolic pressure, the ultrasound systems of the present invention can be further configured to determine and display Doppler in-flow values that can be used to confirm a measurement. In some embodiments, Doppler data can be used to identify the ratio of early mitral inflow velocity (E) to atrial contraction flow velocity (A) and a ratio of E to mitral annular tissue velocity (e′) to estimate left ventricular diastolic function. As shown in FIG. 4, the ultrasound systems of the present invention can be configured to display a representation of Doppler inflow data in combination with the waveforms generated about the spatial characteristics of the various heart chambers, e.g., the left atrium and the left ventricle. Early inflow data 82 can be shown along with late transmittal flow data 84. In some embodiments, a transform of the Doppler inflow data can be performed in order to compare, e.g., via convolution or correlation, the transform to the waveforms of the left atrium and left ventricle. The result of the comparison can provide a way to identify further agreement quality of the calculated static pressure values.

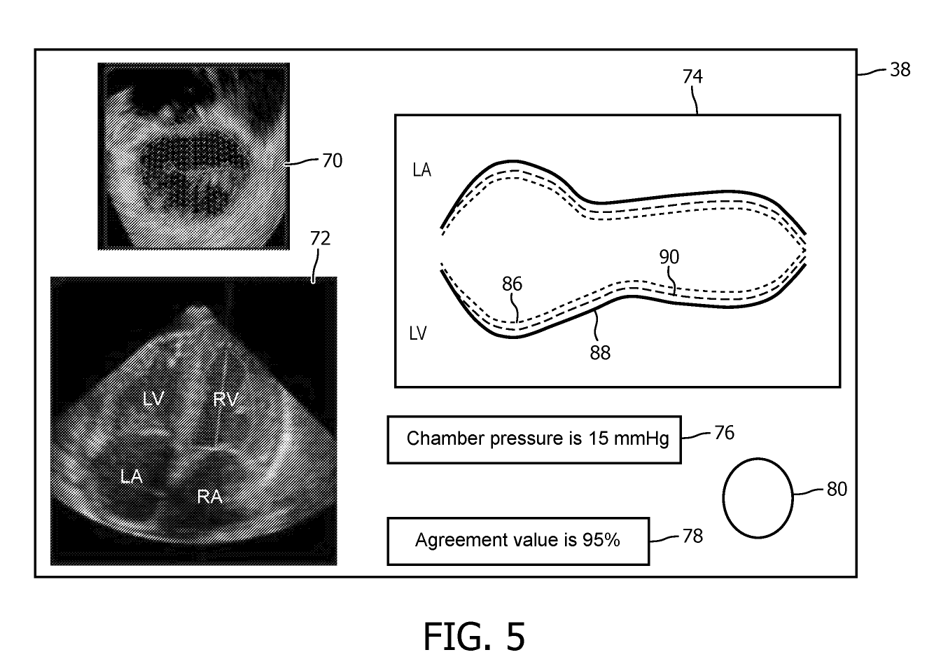

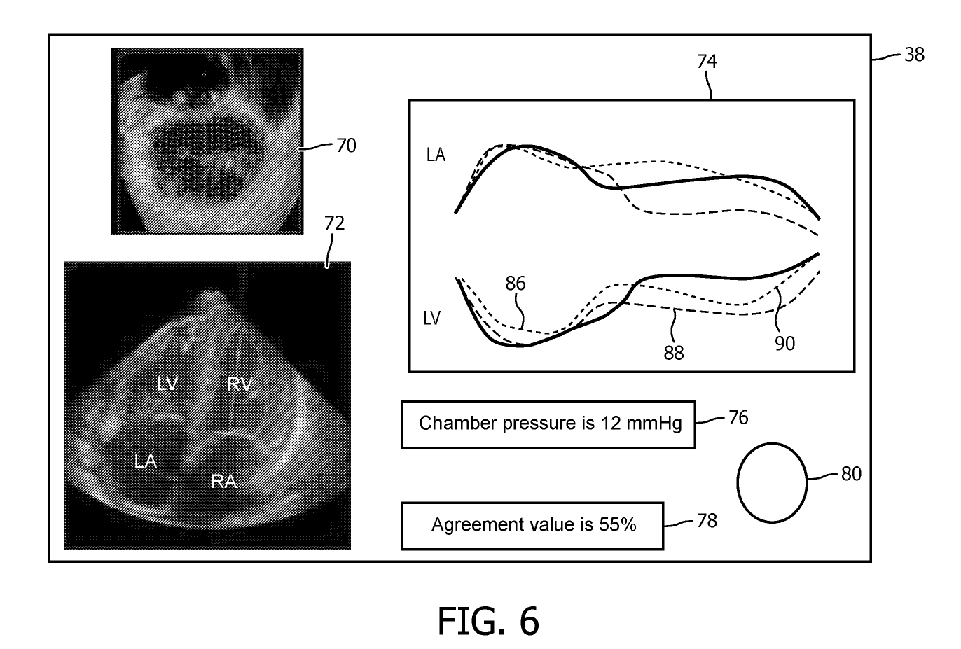

FIGS. 5 and 6 show other examples of displaying data on the display of the ultrasound system to provide better ability to confirm and check the quality of confidence with a particular calculated value of static pressure. In some embodiments, the ultrasound system can display waveforms generated for a variety of methods used to track a spatial characteristic of a heart chamber over time. For example, a waveform from a first method 86 can be overlaid with a waveform from a second method 88 and a third method 90. In FIG. 5, the relative agreement between the different methods is high, indicating that the quality of the calculated static pressure can be used for further diagnosis. In some instances, however, the waveforms from the different methods may not agree. FIG. 6 shows an example of widely varying waveforms 86, 88, and 90 in which an agreement value for the calculated static pressure is at 55%, thus indicating to a user that the analysis is questionable and that new images and/or waveforms should be generated before further diagnosis.

In certain embodiments, the systems and methods of the present invention include configurations in which optimal pressure calculation algorithms can be automatically identified in real-time or during post processing to calculate pressure values. In Kawasaki et al., A novel ultrasound predictor of pulmonary capillary wedge pressure assessed by the combination of left atrial volume and function: A speckle tracking echocardiography study, Journal of Cardiology, Dec. 26, 2014, the total, passive, and active LAEF were defined during a cardiac cycle as (maximum LAV−minimum LAV)/maximum LAV×100%, (maximum LAV−pre-atrial contraction LAV)/maximum LAV×100% and (pre-atrial contraction LAV−minimum LAV)/pre-atrial contraction LAV×100%, respectively. In the patients with chronic AF, total LAEF was substituted for active LAEF because pre-atrial contraction LAV was not present in the patients with AF. As provided herein, the present invention can automatically identify whether total LAEF (mode 1) or active LAEF (mode 2) should be used depending on the waveform collected from the patient. For active LAEF, the LA waveform shape includes a large region of near zero slope at time of pre-atrial contraction. For total LAEF, the LA waveform shape has limited or no region of near zero slope at time of pre-atrial contraction. For mode 1, the pressure estimation algorithm uses only LAVmin and LAVmax. For mode 2, the pressure estimation algorithm uses LAVmin, LAVmax and LAVpre-atrial contraction). For the present invention described herein, the LA volume will be calculated and tracked over time. The system will automatically identify LAVmax as the waveform is generated in real-time or after acquisition of a cineloop capturing the heart over a given time period. After the LAVmax is identified, the system will begin calculating the slope of the LA volume/time waveform. For example, the LA volume over time will be tracked between about 120-180 msec into the heart beat. If the slope of the LA volume over the time period is above a certain threshold, then the waveform indicates that total LAEF will be used for calculating the pressure. However, if the slope of the LA volume over the time period is below a certain threshold, then the active LAEF will be used for calculating the pressure. In some embodiments, the threshold for using the active LAEF mode is less than about 5%, 10%, 15%, or 20%, or between 5% to 20%, between 10% to 20%. That is, if the starting LA volume (e.g., 30 mL) at the beginning of the time period (e.g., 120 msec) is no more than 20% greater than the LA volume (e.g., 25 mL) at the end of the time period (e.g., 180 msec), then active LAEF will be used for the calculations. But, if the starting LA volume (e.g., 30 mL) at the beginning of the time period (e.g., 120 msec) is more than 20% greater than the LA volume (45 mL) at the end of the time period (e.g., 180 msec), then total LAEF will be used for the calculations.

Certain additional advantages and features of this invention may be apparent to those skilled in the art upon studying the disclosure, or may be experienced by persons employing the novel system and method of the present invention. It is to be appreciated that any one of the above embodiments or processes may be combined with one or more other embodiments and/or processes or be separated and/or performed amongst separate devices or device portions in accordance with the present systems, devices and methods.

It should be noted that the various embodiments described herein may be implemented in hardware, software or a combination thereof. The various embodiments and/or components, for example, the modules, or components and controllers therein, also may be implemented as part of one or more computers or microprocessors. The computer or processor may include a computing device, an input device, a display unit and an interface, for example, for accessing the Internet. The computer or processor may include a microprocessor. The microprocessor may be connected to a communication bus, for example, to access a PACS system. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, solid-state thumb drive, and the like. The storage device may also be other similar means for loading computer programs or other instructions into the computer or processor.

As used herein, the term “computer” or “module” or “processor” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of these terms.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

Furthermore, the limitations of the following claims are not written in means-plus-function format and are not intended to be interpreted based on 35 U.S.C. 112, sixth paragraph, unless and until such claim limitations expressly use the phrase “means for” followed by a statement of function devoid of further structure.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. An ultrasound system for automatically determining a static pressure in at least one heart chamber of a patient, comprising:
an image processor configured to receive ultrasound image data and adapted to produce an ultrasound image comprising at least one heart chamber;
a segmentation processor configured to identify a boundary of the at least one heart chamber in the ultrasound image and determine a spatial characteristic of the at least one heart chamber;
a waveform processor configured to generate a waveform representing changes of the spatial characteristic over a time period; and
a display configured to display a representation of the static pressure in the at least one heart chamber of the patient based at least in-part on the waveform.

2. The ultrasound system of claim 1, wherein the at least one chamber comprises a left atrium, a left ventricle, a right atrium, a right ventricle, or a combination thereof.

3. The ultrasound system of claim 1, wherein the system is configured to determine static pressures in at least two heart chambers, and the static pressures comprise right ventricular end diastolic pressure and right ventricle end systolic pressure.

4. The ultrasound system of claim 1, wherein the static pressure in the at least one heart chamber comprises left ventricular end diastolic pressure.

5. The ultrasound system of claim 1, wherein the ultrasound image comprises a standard view of the heart chamber, the standard view comprising a four-chamber view, five-chamber view, three-vessel view, a tracheal view, or a combination thereof.

6. The ultrasound system of claim 1, wherein the boundary of the at least one heart chamber comprises a left ventricle and a left atrium or a right ventricle and a right atrium.

7. The ultrasound system of claim 1, wherein the spatial characteristic comprises a volume, an area, a perimeter, a cavity length, or a strain curve of the at least one heart chamber.

8. The ultrasound system of claim 1, wherein the display is further configured to display the waveform representing changes in the spatial characteristic over the time period.

9. The ultrasound system of claim 1, wherein the display is further configured to display an agreement value determined by comparing waveforms representing two spatial characteristics over the time period, wherein congestive heart failure is diagnosed if the agreement value is above a predetermined threshold.

10. The ultrasound system of claim 1, wherein the display is further configured to display a visual graphic an agreement value determined by comparing waveforms representing two spatial characteristics over the time period, an R-wave generated from Doppler data, and transmittal flow of the heart chamber identified from Doppler data, wherein congestive heart failure is diagnosed if the agreement value is above a predetermined threshold.

11. The ultrasound system of claim 1, wherein the display is further configured to display the ultrasound image along with the waveform representing changes in the spatial characteristic over the time period along with the static pressure.

12. The ultrasound system of claim 1, wherein different representations of the static pressure are determined using a plurality of methods, and the display is further configured to display the resulting static pressure values on the display for user review.

13. The ultrasound system of claim 1, wherein the display is further configured to display waveforms representing changes in the spatial characteristic over the time period for each of the plurality of methods used to determine the different representations of the static pressure.

14. The ultrasound system of claim 1, wherein the system is configured to determine the static pressure using at least one ultrasound image of the at least one heart chamber and the waveform.

15. The ultrasound system of claim 1, wherein the system is configured to automatically determine whether to calculate pressure based on (1) maximum LA volume, pre-atrial contraction LA volume, and minimum LA volume or (2) maximum LA volume and minimum LA volume depending on a slope of the waveform between the LA maximum volume and the LA minimum volume.
